(19) United States
(12) Patent Application Publication
(10) Pub. No.: US 2013/0090369 A1
(43) Pub. Date: Apr. 11, 2013

(54) TRANSFECTION SHEETS AND METHODS OF USE

(71) Applicants: Joe Inoue, Tokyo (JP); Toshihiro Ishizawa, Tokyo (JP)

(72) Inventors: Joe Inoue, Tokyo (JP); Toshihiro Ishizawa, Tokyo (JP)

(73) Assignee: Empire Technology Development LLC

(21) Appl. No.: 13/574,030

(22) PCT Filed: Oct. 6, 2011
(86) PCT No.: PCT/US2011/055087

§ 371 Date: Jul. 19, 2012

(51) Int. Cl. A61K 31/711; G01N 1/28; C12N 15/88; C12N 5/071; A61K 31/7105; A61K 31/713

(52) U.S. Cl. 514/44 A; 514/44 R; 435/320.1; 435/458; 435/375; 427/2.13

(57) ABSTRACT

The present disclosure relates to nucleic acid-lipid compositions for use in delivering nucleic acids to cells in vitro or in vivo. In particular, it relates to the preparation and use of resilient transfection sheets that comprise the nucleic acid-lipid compositions.

### TECHNICAL FIELD

The present disclosure relates generally to methods and compositions for cell transfection. In particular, the present disclosure includes resilient transfection sheets which can be positioned on the cells, tissue or organ to be transfected.

### BACKGROUND

The following description is provided to assist the understanding of the reader. None of the information provided or references cited is admitted to be prior art.

The use of cationic lipids to deliver nucleic acids into cultured cells was first described by Feigner and co-workers (Proc. Nat'l Acad. Sci. 84,7413 (1987)). Subsequently, Behr (Proc. Nat'l Acad. Sci. 86, 6982 (1989)) showed that polycationic lipids also can be effective delivery agents. Large numbers of cationic lipid reagents have now been described and several of these reagents are commercially available, for example, LipofectAmine, LipofectAmine 2000, Fugene, TransfectAm, Lipofectin and DOTAP.

### SUMMARY

Disclosed herein are compositions, methods and kits for cell transfection which include resilient transfection sheets. In one aspect, a composition for cell transfection is provided. In some embodiments, the composition includes one or more cationic lipids, one or more helper lipids, and one or more nucleic acids, wherein the composition is in the form of a resilient sheet. In some embodiments, the one or more helper lipids are neutral lipids. Additionally or alternatively, in some embodiments, the cationic lipid includes an amphiphilic cationic lipid. In some embodiments, the cationic lipid is selected from the group consisting of: 1,2-dioleoyl-1,3-trimethylammonium-propane (DOTAP), 3β-[N-(N′,N′-dimethylaminoehtane)-carbomoyl]cholesterol (DC cholesterol), and combinations thereof. Additionally or alternatively, in some embodiments, the helper lipid selected from the group consisting of: dioleoylphosphatidylethanolamine (DOPE), dipalmitoylphosphatidylcholine (DPPC), distearoylphosphatidylcholine (DSPC), palmitoyloleoyl-phosphatidylcholine (POPC), palmitoyloleoyl-phosphatidylethanolamine (POPE), palmitoyloleyol-phosphatidylglycerol (POPG), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), egg phosphatidylcholine (EPC), or a combination thereof. In some embodiments, the helper lipid is dioleoylphosphatidylethanolamine (DOPE).

In some embodiments, the nucleic acid includes one or more of DNA, RNA, microRNA, siRNA, antisense DNA, plasmid DNA, aptamers, peptide nucleic acid (PNA), and CpG-DNA.

In some embodiments, the molar ratio of cationic lipid to helper lipid is from about 1 to about 0.1-1.0. In some embodiments, the molar ratio of cationic lipid to nucleic acid is about 1 to about 1-1.1. For example, in some embodiments, the resilient sheet comprises DOTAP and DOPE at a ratio of about 1 mole to about 0.1-1 mole.

In some aspects, a method for transfecting a cell is provided. In some embodiments, the method comprises contacting the cell with one or more of the resilient sheet compositions described above. In some embodiments, the cell is in vitro. In some embodiments, the cells is in vivo. Additionally or alternatively, in some embodiments, the cell is a mammalian cell. Additionally or alternatively, in some embodiments, the cell is part of an organ.

In some aspects, a method of treating a subject with a therapeutic nucleic acid is provided. In some embodiments, the method includes contacting the subject with the resilient sheet as described above. In some embodiments, the nucleic acid of the resilient sheet comprises a therapeutic nucleic acid. For example, in some embodiments, the therapeutic nucleic acids includes one or more of a therapeutic siRNA, microRNA, an aptamer, or a combination thereof. Additionally or alternatively, in some embodiments, the therapeutic nucleic acids encodes a therapeutic polypeptide. Additionally or alternatively, the nucleic acid includes one or more of DNA, RNA, microRNA, siRNA, antisense DNA, plasmid DNA, aptamers, peptide nucleic acid (PNA), and CpG-DNA. Additionally or alternatively, in some embodiments, the resilient sheet contacts an organ of the subject. Additionally or alternatively, in some embodiments, the resilient sheet contacts one or more of muscle tissue, epithelial tissue, connective tissue and nervous tissue of the subject. Additionally or alternatively, in some embodiments, the resilient sheet contacts one or more of hematopoietic cells, epithelial cells and immune cell precursors of the subject.

In some aspects, a method of making a resilient sheet for cell transfection is provided. In some embodiments, the methods include combining a cationic lipid, a helper lipid and a nucleic acid to form a mixture, casting the mixture onto a support surface, and drying the cast solution thereby forming a resilient sheet. In some embodiments, the method includes cutting the dried resilient sheet to a desired shape and or size. In some embodiments, the ratio of cationic lipid to helper lipid is from about 1 to about 0.1-1.0. In some embodiments, the ratio of cationic lipid to nucleic acid is about 1 to about 1-1.1. In some embodiments, drying comprises evaporation at room temperature under ambient pressure. In some embodiments, the cationic lipid is selected from the group consisting of: 1,2-dioleoyl-1,3-trimethylammonium-propane (DOTAP), 3β-[N-(N′,N′-dimethylaminoehtane)-carbomoyl]cholesterol (DC cholesterol), and combinations thereof. In some embodiments, the helper lipid is selected from the group consisting of: dipalmitoylphosphatidylcholine (DPPC), distearoylphosphatidylcholine (DSPC), palmitoyloleoyl-phosphatidylcholine (POPC), palmitoyloleoyl-phosphatidylethanolamine (POPE), palmitoyloleyol-phosphatidylglycerol (POPG), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), egg phosphatidylcholine (EPC) or a combination thereof. In some embodiments, the nucleic acid comprises one or more of: DNA, RNA, microRNA, siRNA, antisense DNA, plasmid DNA, aptamers and CpG-DNA.

In some aspects, kit for transfection are provided. In some embodiments, the kits include one or more resilient sheets as described above. Optionally, in some embodiments, the kits include instructions for use, such as for use in transfection.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF THE DRAWINGS

FIG. 1 is a flow chart illustrating the steps of preparing resilient transfection sheets (gene transfer sheets), and illustrative in vitro and in vivo applications of the sheets.

FIG. 2 shows CpG-DNA-induced IL-6 production in BALB/c peritoneal macrophages.

### DETAILED DESCRIPTION

Disclosed herein are methods and compositions related to the introduction of nucleic acids into cells (transfection), both in vitro and in vivo. In one aspect, the methods and compositions include resilient sheets, comprising cationic lipids, helper lipids and nucleic acids. The resilient sheets can be attached directly to the target site to transfer nucleic acids easily and efficiently. In addition to efficient nucleic acid transfer, the resilient sheets allow for easy and inexpensive storage and handling of lipid-nucleic acid compositions.

### I. DEFINITIONS

The following terms are used herein, the definitions of which are provided for guidance.

As used herein, the singular forms “a,” “an,” and “the” designate both the singular and the plural, unless expressly stated to designate the singular only.

The term “about” and the use of ranges in general, whether or not qualified by the term about, means that the number comprehended is not limited to the exact number set forth herein, and is intended to refer to ranges substantially within the quoted range while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein “subject” and “patient” are used interchangeably and refer to an animal, for example, a member of any vertebrate species. The methods of the presently disclosed subject matter are particularly useful for warm-blooded vertebrates. Mammals such as humans, as well as those mammals of importance due to being endangered, of economic importance (animals raised on farms for consumption by humans) and/or social importance (animals kept as pets or in zoos) to humans. In particular embodiments, the subject is a human. In some embodiments, the subject is not a human.

As used herein, the terms “effective amount” or “therapeutically effective amount” or “pharmaceutically effective amount” refer to a quantity sufficient to achieve a desired therapeutic and/or prophylactic effect, e.g., an amount which results in the prevention of, disease, condition and symptoms thereof. In the context of therapeutic or prophylactic applications, the amount of a composition administered to the subject will depend on the type and severity of the disease and on the characteristics of the individual, such as general health, age, sex, body weight and tolerance to drugs. It will also depend on the degree, severity and type of disease or condition. The skilled artisan will be able to determine appropriate dosages depending on these and other factors. Multiple therapeutic compositions or compounds can also be administered.

As used herein, the term “cationic lipid” refers to a lipid which has a cationic, or positive, charge at physiologic pH. It is well known in the art that cationic lipids form a variety of polymorphic phases when dispersed in aqueous solution, such as liposomes and micelles, and may be used to facilitate the transport of nucleic acids into cells (transfection). Whether a cationic lipid occurs primarily as a liposome or a micelle can be manipulated by methods known in the art; for example, a freezing and thawing of cationic lipids in aqueous solution will encourage formation of liposomes, rather than micelles.

In illustrative embodiments, cationic lipids have a cationic group, such as a quaternary amine group, and one or more lipophilic groups, such as saturated or unsaturated alkyl groups having from about 6 to 30 carbon atoms. The structure of cationic lipids has a substantial impact on their transfection efficiency. Cationic lipids generally include four functional domains: a hydrophilic head group, a linker, a backbone domain, and a hydrophobic domain. The precise structure of the hydrophobic domain determines the phase transition temperature and fluidity of the lipid bilayer, and influences the stability of liposomes. It also influences the extent to which exogenous nucleic acids are protected from endogenous nucleases, whether the exogenous nucleic acid escapes the endosome, and whether the exogenous nucleic acid translocates to the nucleus of the transfected cell. The toxicity level of the lipid is also influenced by the hydrophobic domain. Lipids typically used for gene therapy are classified according to the structure of the hydrophobic domain. Illustrative hydrophobic domain types are 1) an aliphatic chain, 2) a steroid domain, and 3) a fluorinated domain. In illustrative embodiments, the cationic lipid includes an amphiphilic cationic lipid.

Non-limiting examples of cationic lipids suitable for use in the compositions and methods disclosed herein include commercially available cationic lipids, for example 1,2-di-O-octadecenyl-3-trimethylammonium propane (DOTMA), available from Life Technologies Inc., (LTI), Gaithersburg, MD under the trademarked name of LIPOFECTIN™, and dime-thyldioctadecyl ammonium bromide (DDAB), available from Boehringer-Mannheim, Indianapolis, Ind. Non-limiting examples of commercially available cationic lipid compositions include phosphatide carriers such as FuGENE® 6 Transfection Reagent (Roche Diagnostics K.K.), LIPOFECTAMINE™ (Life Technologies, Japan, Ltd.), TransfectAm®, Lipofectin®, and 1,2-dioleoyl-3-trimethylammonium-propane (DOTAP).

Additional non-limiting examples of cationic lipids include 1-[2-(oleoyloxy)ethyl]-2-oleyl-3-(2-hydroxyethyl)imidazolinium chloride (DOTIM; also called BODAI), 1,2-dimyristyloxypropyl-3-dimethylhydroxyethyl ammonium bromide (DMRIE), 1,2-dimyristoyl-sn-glycero-3-ethylphosphocholine (EDMPC) (commercially available from Avanti Polar Lipids, Alabaster, Ala.), 3β-[N-(N′,N′-dimethylaminoethane)-carbamoyl]cholesterol hydrochloride (DC-cholesterol), dioctadecylamidoglycyl-spermine (DOGS), MBOP (also called MeBOP or MBN222). Cholesterol derivatives having a cationic polar head group are also encompassed within the definition.

Other cationic lipids include the class of lipids known as tetramethyltetraalkyl spermine analogs. Lipids of this type include tetramethyltetralaurylspermine, tetramethyltetramyristylspermine, tetramethyltetrapalmitoyispermine, and tetramethyltetraoleoylspermine. The following lipids, obtained from LTI are of the tetramethyltetraalkyl spermine class, with the alkyl groups containing fatty acid chains of length longer than oleic acid. These lipids are denoted as LTI lipids 4251-781-1, 4251-106-3, 4518-52, D304-200, 4521-52-3, 4251-106-4, 4251-781-2, 4518-53, 4518-31, 4519-30,4519-34, and 2518-111.

Other suitable cationic lipid compounds are described in the literature. See, for example, Stamatatos et al., 1988, Biochemistry 27, 3917-3925 and Eibl, et al., 1979, Biophysical Chemistry 10, 261-271.

In addition, suitable cationic lipids can be synthesized as described in the literature; see, for example, Feigner et al., 1987, PNAS 84 7413-7417 (DOTAP); Douar et al, 1996, Gene Ther 3(9), 789-796 (Lipid 67); Wheeler et al., 1996, Biochim Biophys Acta 1280(1), 1-11 (DMRIE); McLean et al., 1997 Am J Physiol 273, H387-404 (DOTIM); and Hofland et al., 1997, Pharm Res 14(6), 742-749 (DOSPA).

The compositions and methods disclosed herein may include a single cationic lipid or a combination of one or more cationic lipids. The compositions and methods disclosed herein may include a single nucleic acid or a combination of one or more therapeutic nucleic acids.

Cationic lipid compositions suitable for use in the present invention include lipid compositions including one type of lipid, or lipid compositions including more than one type of lipid. In illustrative embodiments, when more than one type of lipid is present in a lipid composition, the net charge of the lipid composition is positive. In illustrative embodiments, the lipid composition includes cationic, anionic, and/or neutral lipids, and the net charge of the composition is positive.

As used herein, the term “neutral lipid” refers to a lipid having no charge at physiological pH. Examples of neutral lipids include triglycerides made of 3 fatty acids linked to a glycerol, with no polar group, such as present in phospholipids. Additional illustrative, non-limiting neutral lipids include dipalmitoylphosphatidylcholine (DPPC), distearoylphosphatidylcholine (DSPC), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), and egg phosphatidylcholine (EPC). The compositions and methods disclosed herein may include a single neutral lipid or a combination of one or more neutral lipids.

As used herein, the term “helper lipid” refers to lipids which improve transfection efficiency and/or provide a functional or a structural advantage in the formation, use or storage of the resilient sheets. In some embodiments, helper lipids are neutral lipids. It is well known in the art that the inclusion of a helper lipid in a lipid-nucleic acid composition improves the efficiency of transfection. Not wishing to be bound by theory, it is believed that transfection efficiency using cationic lipids alone is low due to the inability of exogenous nucleic acids to escape endosomes. It is thought that helper lipids destabilize endosomal membranes so as to facilitate the escape of nucleic acids from the endosome. Unsaturated phosphatidylethanolamines (PEs) are commonly used as helper lipids. Not wishing to be bound by theory, it is believed that the effectiveness of unsaturated PEs, such as DOPE, lies in their propensity to form non-bilayer structures similar to membrane fusion intermediates. This property is thought to facilitate the fusion of cationic liposomes to cell membranes. Additionally or alternatively, helper lipids facilitate the release of the nucleic acid from the resilient sheet, thereby allowing transfection of the desired target. Without wishing to be bound by theory, it is thought that the helper lipid reduces the binding force between the cationic lipids and the nucleic acids. Thus, in some embodiments, the resilient sheets which include one or more helper lipids can be broken down with a change of pH and/or temperature, etc., thereby releasing the nucleic acids for transfection. In some embodiments, helper lipids which are pH and/or temperature sensitive are used.

Non-limiting examples of helper lipids include dipalmitoylphosphatidylcholine (DPPC), distearoylphosphatidylcholine (DSPC), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dipalmitoyl-phosphatidylethanolamine (DPPE), dirnyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), egg phosphatidylcholine (EPC), and mixtures thereof.

In illustrative embodiments, the helper lipid is 1,2-dilauroyl-sn-glycero-3-phosphoethanolamine (DLPE) and/or 1,2-diphytanoyl-sn-glycero-3-phosphoethanolamine (DiPPE). In an illustrative embodiment, cholesterol is a helper lipid. In an illustrative embodiment, DOPC is a helper lipid. In another illustrative embodiment, DOPE is a helper lipid. The compositions and methods disclosed herein may include a single helper lipid or a combination of one or more helper lipids.

In some embodiments, the nucleic acid-lipid composition of the resilient sheets is sensitive to temperature and/or pH. In illustrative embodiments, this sensitivity is conferred by physical properties of the helper lipid. For example, in illustrative embodiments, the lipid chain melting temperature of the helper lipid is at or near the ambient temperature of the transfection target such that contact with the transfection target causes the helper lipid to undergo a phase transition. In some embodiments, the ambient temperature of the transfection target is 25° C. In some embodiments, the ambient temperature of the transfection target is 37° C. In illustrative embodiments, the lipid comprises a lipid having an unsaturated aliphatic acid chain, such as palmitic acid or stearic acid. In illustrative embodiments, the lipid is nonpolar. In illustrative embodiments, such phase transition alters the structural conformation of the lipid component of the nucleic acid-lipid composition of the resilient sheet. In illustrative embodiments, the conformational change promotes release of the nucleic acid and facilitates transfer to the nucleic acid to the transfection target. For example, in illustrative in vivo uses of the transfection sheet, rapid warming of the lipid composition following contact with a target organ or tissue causes a phase transition in the helper lipid that facilitates the release of nucleic acids from the transfection sheet and transfer of the nucleic acids to the target. In illustrative embodiments, the helper lipid phase transition is induced by the pH of the transformation target. In illustrative embodiments, the helper lipid is dioleoylphosphatidylethanolamine (DOPE). In illustrative embodiments, the DOPE undergoes a phase transition at about pH 6.5 to a hexagonal II conformation and begins membrane fusion. In illustrative embodiments, the helper lipid phase transition is induced by a combination of temperature and pH. In illustrative embodiments, the pH-sensitive helper lipid is dilauroylphosphatidylethanolamnine (DLPE), dimyristoylphosphatidyl-ethanolamine (DMPE), dipalmitoylphosphatidylethanolamine (DPPE), or cholesterol.

As used herein, the terms “resilient sheet,” “transfection sheet,” “nucleic acid transfer sheet” or “gene transfer sheet” refer to a composition comprising lipids and nucleic acids which have sufficient structural integrity so as to be manipulated, moved, etc. without requiring a supportive backing. In illustrative embodiments, the lipid comprises a cationic lipid, a helper lipid, or a mixture thereof. In illustrative embodiments, the cationic lipid comprises a mixture of cationic lipids. In illustrative embodiments, the helper lipid comprises a mixture of helper lipids. In illustrative embodiments, the nucleic acid is DNA, RNA, or a mixture thereof.

The resilient sheets disclosed herein are typically prepared by casting a lipid-nucleic acid composition onto a support surface and allowing the composition to dry. In some embodiments, the lipid-nucleic acid composition is cast onto a sterile, relatively flat support surface. Illustrative non-limiting support surfaces include glass, plastics, stainless steel, aluminum, ceramic, etc. In an illustrative embodiment, the surface is a flexible medium such as a Teflon™ sheet. In an illustrative embodiment, the support surface is a flexible medium such as cellophane. In illustrative embodiments, the support surface is a cell culture dish. In illustrative embodiments, the support surface includes any medium compatible with the intended use of the lipid-nucleic acid composition. Once the sheets are dried, they are removed from the support surface and stored for later use, or are applied directly to the transfection target.

In illustrative embodiments, the sheets are air-dried at room-temperature. In illustrative embodiments, the sheets are dried at a temperature greater than room-temperature.

In some embodiments, drying time is dependent on drying conditions (e.g., temperature, vacuum, humidity, etc.) the size of the sheet and/or the amount of solvent used. In illustrative embodiments, the sheets are dried for approximately 30 minutes, approximately 60 minutes, approximately 2 hours, approximately 3 hours or approximately 6 hours. In illustrative embodiments, the sheets are dried overnight. In illustrative embodiments, the dried sheets are removed from the solid support with a forceps. In illustrative embodiments, the sheets are stored in a sealed container under nitrogen gas. In illustrative embodiments, the sheets are stored at or below minus 20° C. In illustrative embodiments, the sheets are stored at room temperature.

The resilient sheets described herein are typically flexible, relatively dry and can be easily moved and positioned for use, for example with tweezers, forceps or fingers. Resilient sheets can be of any shape and size that is convenient for the intended use. For example, if a sheet is to be used for transfection in vitro in a round cell culture dish, the sheet can be formed, shaped or cut to cover the bottom of the round dish. Additionally or alternatively, the lipid-nucleic acid composition can be cast directly in the culture dish, and the cells can be added after the composition has dried. If a sheet is to be used in vivo, for example to transfect the cells of an organ, part of an organ, or specific region of a tissue, the sheet may be formed in or cut to an advantageous shape and size for that particular application.

The term “nucleic acid” as used herein refers to a polymer or oligomer composed of nucleotide units (ribonucleotides, deoxyribonucleotides or related structural variants or synthetic analogs thereof) linked via phosphodiester bonds (or related structural variants or synthetic analogs thereof). Thus, the term refers to a nucleotide polymer in which the nucleotides and the linkages between them are naturally occurring (DNA or RNA), as well as various analogs, for example and without limitation, peptide-nucleic acids (PNAs), phosphoramidates, phosphorothioates, methyl phosphonates, 2-O-methyl ribonucleic acids, and the like.

The nucleic acid may be in any physical form, e.g., linear, circular or supercoiled; single-stranded, double-, triple-, or quadruple-stranded; and further including those having naturally occurring nitrogenous bases and phosphodiester linkages as well as non-naturally occurring bases and linkages, e.g. for stabilization purposes. For example, in illustrative embodiments, the nucleic acid it is in the form of supercoiled plasmid DNA. Plasmid DNA is conveniently used for DNA transfections since, in general, it can be produced in large quantities by growing and purifying it from bacterial cells. The nucleic acid may be, without limitation, an antisense molecule, or may be a double stranded RNA molecule of the type used for inhibiting gene expression by RNA interference. The nucleic acid may be a short interfering double stranded RNA molecule (siRNA). In illustrative embodiments, the DNA is viral DNA, retroviral DNA, cosmid DNA, or linear DNA.

Typically, the nucleic acid is sufficiently negatively charged to form a lipid aggregate, liposome, or liposome-like complex when admixed with the cationic lipid.

As used herein the term “transfection” refers to the delivery of exogenous nucleic acid molecules to a cell, either in vivo or in vitro, whereby the nucleic acid is taken up by the cell and is functional within the cell. A cell that has taken up the exogenous nucleic acid is referred to as a “host cell” or “transfected cell.” A nucleic acid is functional within a host cell when it is capable of functioning as intended. For example, in illustrative embodiments, the exogenous nucleic acid will include an expression cassette which includes DNA encoding a protein of interest, with appropriate regulatory elements. Such nucleic acid will function as intended if the DNA is transcribed and translated by the host cell, thereby causing the host cell to produce the encoded protein. In illustrative embodiments, the transfected nucleic acid encodes a protein which is lacking, produced in insufficient quantity, or produced in a less than fully active form in the host cell. In other illustrative embodiments, the transfected nucleic acid encodes a protein that is secreted from the cell, wherein the expressed and secreted protein may have an effect on cells other than the transfected cell. Other non-limiting examples of exogenous nucleic acids to be delivered to cells include, e.g., nucleic acids as drugs, for example, aptamers, antisense DNA or RNA, mRNA, CpG-DNA, siRNA, peptide nucleic acids (PNAs), or ribozymes. Nucleic acids of interest also include DNA coding for a cellular factor which, when expressed, activates the expression of an endogenous gene. In illustrative embodiments, the DNA is designed to integrate into the genome of the host cell and thereafter be expressed. In some embodiments, the DNA is designed to integrate into the genome of the host cell and thereby disrupt a host gene. In illustrative embodiments, the nucleic acid is a DNA or RNA mimic. In illustrative embodiments, the mimic is a peptide nucleic acid (PNA).

As used herein, “transfection efficiency” refers to the relative number of cells of the total within a cell population that are transfected with a particular nucleic acid composition and/or to the level of exogenous nucleic acid expression obtained in the transfected cells. Those of skill in the art will understand that through the use of regulatory control elements such as promoters, enhancers and the like, the level of exogenous gene expression in a host cell can be modulated. The transfection efficiency necessary or desirable for a given purpose will depend on the purpose, for example the disease indication for which treatment is intended, and on the level of gene expression obtained in the transfected cells.

As used herein, the term “complexed with” and “complexed to” are equivalent and refer to any method by which a nucleic acid molecule interacts with (e.g. binds to, comes into contact with, adheres to) a cationic lipid. Such an interaction can include, but is not limited to encapsulation of a nucleic acid molecule into a cationic liposome, association of a nucleic acid molecule and cationic lipid characterized by non-covalent, ionic charge interactions, and other types of associations between nucleic acid molecules and cationic lipids known by those skilled in the art.

### II. TRANSFECTION SHEETS

A. General

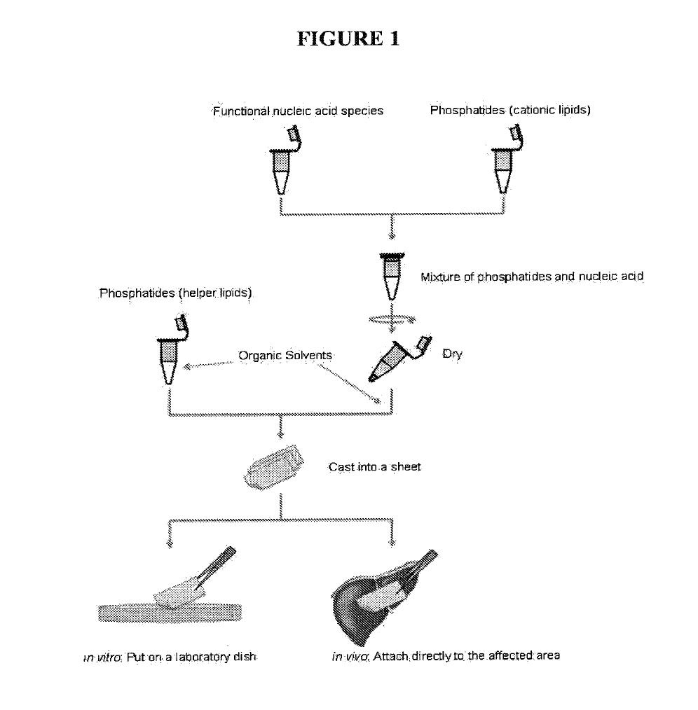

The resilient sheets disclosed herein have numerous advantages over conventional transfection compositions and methods. In illustrative embodiments, the resilient sheets maintain sufficient structural integrity to be moved independently of any sort of a base of support. For example, tweezers or fingers can be used to manipulate, position or move the sheets. In some embodiments, the sheet is cut to an appropriate size and/or shape for its intended use. In addition, in some embodiments, the sheets limit nucleic acid diffusion in the body or in solution (e.g., in cell culture medium). As shown in FIG. 1, in illustrative embodiments, the sheet is placed directly on the target site, e.g., the sheet is positioned on a tissue or organ for in vivo, ex vivo, or in vitro use, or is positioned in a laboratory dish for in vitro transfections. Such simplicity of use reduces operational errors, and increases the efficiency of nucleic acid transfer.

In addition, in illustrative embodiments, the sheets are optionally stored long term at room temperature, in dry conditions (e.g., the sheets do not have to be kept wet or moist). Vacuum-packing or otherwise storing the sheets away from oxygen allows for even longer term storage. For example, in illustrative embodiments, the sheets can be stored for 3-5 days, 1-2 weeks, 1-2 months, 3-6 months, 6 months to a year, or for a year or more at room temperature under dry conditions. For example, there is no need for cold storage during storage or shipping, which can significantly reduce costs involved with product use.

In addition, the transfection sheets disclosed herein have the potential to replace gene transfer carriers currently used for in vitro studies. The transfection sheets are easy to manufacture, store, and use. By simply placing a transfection sheet in a cell culture dish, efficient transfection of cells can be expected.

With respect to in vivo embodiments, the sheets can be used to treat patients by, for example, transferring nucleic acid drugs, such as siRNA and antisense DNA to inhibit gene expression, and/or expression vectors to supplement a endogenous levels of a particular molecule. The sheets may also be used to deliver aptamers, which can specifically bind to and inhibit certain protein targets. In some embodiments, the nucleic acid comprises a nucleic acid vaccine.

B. Compositions

The resilient transfection sheets disclosed herein are typically formed from cationic lipids and nucleic acids. In illustrative embodiments, helper lipids are optionally included. Negatively charged nucleic acids interact with cationic lipids to form a nucleic acid/lipid complex. To prepare the transfection sheets, cationic lipids are mixed with nucleic acid in solution, at concentrations and ratios optimized for the target cells to be transfected. Alterations in the lipid formulation and mode of delivery allow preferential delivery of nucleic acids to particular tissues in vivo.

Typically, the ratio of nucleic acid to cationic lipid is from about 1 to 1.1 mole of nucleic acid per 1 mole of cationic lipid. In illustrative embodiments, the ratio of nucleic acid to cationic lipid is from about 0.1-1.5 mole of nucleic acid to about 3 moles of cationic lipid, from about 0.1-1.5 mole of nucleic acid to about 2 moles of cationic lipid, from about 0.1-1.5 mole of nucleic acid to about 1 moles of cationic lipid, from about 0.5-1.2 mole of nucleic acid to about 3 moles of cationic lipid, from about 0.5-1.2 mole of nucleic acid to about 2 moles of cationic lipid, from about 0.5-1.2 mole of nucleic acid to about 1 mole of cationic lipid, from about 1-1.1 mole of nucleic acid to about 3 moles of cationic lipid, or from about 1-1.1 mole of nucleic acid to about 2 moles of cationic lipid.

Helper lipids are optionally included in the compositions and methods described herein. In some embodiments, the helper lipid is dioleoylphosphatidylethanolamine (DOPE). DOPE has been shown to improve transfection efficiencies in vitro when used in conjunction with a number of different cationic lipids. It has been commonly believed that DOPE improved transfection by making the liposomes more fusogenic, thereby improving either fusion with the plasma membrane, fusion with the endosomal membrane, or both. Recent reports have also shown improved in vivo transfection efficiencies using cholesterol as the helper lipid. Other helper lipids include, without limitation, DLPE and DiPPE. In some embodiments, one or more helper lipids sensitive to temperature and/or pH are included. The inclusion of such helper lipids in the transfection sheets allows the sheet to collapse or dissolve with a change in temperature or pH (e.g., after the sheet is positioned on the target cells or target tissue). As noted above, helper lipids facilitate the release of the nucleic acid from the resilient sheet, thereby allowing, enhancing and promoting transfection of the desired target.

In embodiments that include a helper lipid, the molar ratio of helper lipid to cationic lipid is from about 0.05 to about 3, from about 0.1 to about 3, from about 0.2 to about 3, from about 1 to about 3, from about 1.1 to about 3, from about 1.5 to about 3, from about 2 to about 3, from about 0.05 to about 2, from about 0.1 to about 2, from about 0.2 to about 2, from about 1 to about 2, from about 1.1 to about 2, from about 1.5 to about 2, from about 0.05 to about 1, from about 0.1 to about 1, from about 0.2 to about 1, from about 1 to about 1. Additionally or alternatively, in some embodiments, the molar ratio of helper lipid to cationic lipid is about 0.05-1.5 mole helper lipid to about 1 mole cationic lipid, about 0.1-1 mole helper lipid to about 1 mole cationic lipid, about 0.5-1 mole helper lipid to about 1 mole cationic lipid, about 0.7-1 mole helper lipid to about 1 mole cationic lipid. In one embodiment, the molar ratio of helper lipid to cationic lipid is about 0.1-1 mole helper lipid to about 1 mole cationic lipid.

C. Preparation

The resilient transfection sheets described herein are generally prepared by casting a nucleic acid-lipid composition onto a relatively flat, solid (e.g., relatively non-porous) surface. In some embodiments, the surface is sterile. In some embodiments, the surface is flexible. In an illustrative embodiment, the surface is a Teflon™ sheet. In an illustrative embodiment, the surface is a cellophane sheet. In an illustrative embodiment, the surface is glass, stainless steel, plastic, or any medium that will hold the liquid nucleic acid-lipid composition until it dries.

The nucleic acid-lipid compositions described herein generally include nucleic acids intended to be delivered to cells, organs, or tissues in vitro, in vivo, or ex vivo, one or more cationic lipids, and one or more neutral helper lipids. Non-limiting examples of cationic lipids suitable for the preparation of resilient transfection sheets are N,N-dioleyl-N,N-dimethylammonium chloride (DODAC), 1,2-dioleyloxy-N,N-dimethylaminopropane (DODMA), 1,2-distearyloxy-N,N-dimethylaminopropane (DSDMA), N-(1-(2,3-dioleyloxy)propyl)-N,N,N-trimethylammonium chloride (DOTMA), N,N-distearyl-N,N-dimethylammonium bromide (DDAB), N-(1-(2,3-dioleoyloxy)propyl)-N,N,N-trimethylammonium chloride (DOTAP), 3-(N—(N′,N′-dimethylaminoethane)-carbamoyl)cholesterol (DC-Chol), N-(1,2-dimyristyloxyprop-3-yl)-N,N-dimethyl-N-hydroxyethyl ammonium bromide (DMRIE),2,3-dioleyloxy-N-[2(spermine-carboxamido)ethyl]-N,N-dimet-hyl-1-propanaminiumtrifluoroacetate (DOSPA), dioctadecylamidoglycyl spermine (DOGS),3-dimethylamino-2-(cholest-5-en-3-beta-oxybutan-4-oxy)-1-(cis,cis-9,12-octadecadienoxy)propane (CLinDMA), 2-[5′-(cholest-5-en-3.beta.-oxy)-3′-oxapentoxy)-3-dimethyl-1-(cis,cis-9′,-1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 1,2-N,N′-Dilinoleylcarbamyl-3-dimethylaminopropane (DLincarbDAP), 1,2-Dilinoleoylcarbamyl-3-dimethylaminopropane (DLinCDAP), and mixtures thereof. Additionally, a number of commercial preparations of cationic lipids are available and can be used in the present invention. These include, e.g., LIPOFECTIN™ (GIBCO/BRL, Grand Island, N.Y., USA); LIPOFECTAMINE™ (GIBCO/BRL); and TRANSFECTAM™ (Promega Corp.).

In the preparation of the resilient transfection sheets described herein, in some embodiments, stock solutions of nucleic acid and cationic lipids are combined in aqueous solution, mixed well, and then allowed to stand for a period of 30 to 60 minutes. In some embodiments, during this period, nucleic acid-lipid complexes form and may be visible as a white precipitate. At the conclusion of 30-60 minutes, in some embodiments, the sample is centrifuged at 1,000×g for 5 minutes, the supernatant removed, and the pellet allowed to dry. In some embodiments, the pellet is then resuspended in an organic solvent.

Non-limiting examples of solvents suitable for the preparation the resilient transfection sheets described herein are hexane, methanol, ethanol, chloroform, and the like. Those of skill in the art will understand that the selection of solvent may depend on the specific lipid content of the nucleic acid-lipid complex. Those of skill in the art will further understand that organic solvents used for the preparation of resilient transfection sheets will evaporate upon drying of the sheet such that no additional steps are required to remove the solvents from the end product prior to use of the resilient transfection sheets.

Following resuspension of the acid-lipid complex, a neutral helper lipid is optionally included from a prepared stock solution. Non-limiting examples of helper lipids that are suitable for preparation of the resilient transfection sheets described herein are dipalmitoylphosphatidylcholine (DPPC), distearoylphosphatidylcholine (DSPC), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), egg phosphatidylcholine (EPC), and mixtures thereof. In certain preferred embodiments, the phospholipid is DPPC, DSPC, or mixtures thereof.

Following addition of the helper lipid, the nucleic acid-lipid composition is cast onto support surface and allowed to dry. In an illustrative embodiment, the support surface is a Teflon™ sheet. In an illustrative embodiment, the support surface is a cellophane sheet. In an illustrative embodiment, the support surface is glass, stainless steel or plastic. In illustrative embodiments, drying is performed by evaporation of the solvent at room temperature, under ambient pressure. In illustrative embodiments, drying is performed under vacuum. Additionally or alternatively, in some embodiments, drying is performed at a temperature greater than or less than room temperature. In illustrative embodiments, drying is performed for about 1, about 2, about 3, about 4, about 5, about 6, about 7, about 8, about 9, or about 10 hours. In illustrative embodiments, drying is performed overnight. In illustrative embodiments, drying is performed for a period of time sufficient to remove all or essentially all organic solvent from the nucleic acid-lipid composition. In illustrative embodiments, drying time depends on the specific content of the nucleic acid composition and the organic solvent used. In some embodiments, drying time is sufficient to yield a resilient (e.g., manipulatable) sheet to form.

Additionally or alternatively, the nucleic acid-lipid composition may be cast directly onto a surface on which cells will be cultured in vitro. In an illustrative embodiment, the surface is the bottom of a tissue culture plate or tissue culture well.

Additionally or alternatively, in some embodiments, the resilient sheets are formed as layers, with each layer comprising the same or different concentration or type of nucleic acid. Additionally or alternatively, in some embodiments the different layers include the same or different lipid compositions. In embodiments having different lipid compositions in each layer, the nucleic acid release properties of the lipid layers differs. In some embodiments, nucleic acid release rate is roughly proportional to the amount of helper lipid (e.g., more helper lipid results in faster release). Thus, in some embodiments, different nucleic acids can be delivered to the same target cells, tissues or organs sequentially or simultaneously by applying a layered resilient sheet.

D. Methods

In vivo: The present disclosure provides methods for the transfection of cellular targets in vivo. In illustrative embodiments, the in vivo transformation target is an organ or tissue of a subject. In illustrative embodiments, the transfection sheet contacts the organ or tissue of the subject and the nucleic acid component of the nucleic acid-lipid composition is transferred to cells of the organ or tissue. In illustrative embodiments, the transfection sheet contacts one or more of muscle tissue, epithelial tissue, connective tissue and nervous tissue of the subject. In illustrative embodiments, the transfection sheet contacts one or more of hematopoietic cells, epithelial cells and immune cell precursors of the subject. In illustrative embodiments, the transfection sheet contacts any cells, tissues, or organs of the subject that are targets for transfection.

In illustrative embodiments, the transfection sheet is hydrated upon contact with the target tissue or organ without the need for a hydration step prior to application of the sheet to the target. In illustrative embodiments, hydration is accomplished by contact with cell culture media. In illustrative embodiments, hydration is accomplished by contact with bodily fluids associated with tissues or organs. In some embodiments, hydration is accomplished by contact with media used to preserve a tissue or organ ex vivo. In illustrative embodiments, the pH and or temperature sensitivity of the lipid component of the resilient sheet results in a conformation change in the lipid component (e.g., the helper lipid) upon contact with the target organ or tissue that facilitates the transfer of nucleic acids to the target. For example, in illustrative embodiments, the melt temperature of the helper lipid is at or near the ambient temperature of the target organ or tissue. Warming of the resilient sheet composition following contact with a target organ or tissue causes a phase transition in the helper lipid that facilitates the release of nucleic acids from the transfection sheet and transfer of the nucleic acids to the target. In illustrative embodiments, the helper lipid phase transition is induced by the physiological pH of the transformation target. In illustrative embodiments, the helper lipid phase transition is induced by a combination of temperature and pH of the transformation target.

In vitro: The present disclosure provides methods for the transfection of cellular targets in vitro. In illustrative embodiments, the in vitro transfection target is cultured cells, tissues, or organs. In illustrative embodiments, the cells, tissues, or organs are derived from one or more of muscle, epithelial, connective or nervous tissue cell types. In illustrative embodiments, the transfection target is a primary cell line. In illustrative embodiments, the target is an immortalized cell line. In illustrative embodiments, the in vitro transfection target is a tissue or organ in culture. In some embodiments, the tissue or organ is removed for transplant.

In illustrative embodiments, the target cells are grown in tissue culture plates or wells. In illustrative embodiments, the resilient sheet is cut to a size corresponding to the surface area of the culture dish, and placed in the dish prior to the addition of cells. In illustrative embodiments, the transfection sheet is cast directly into the culture dish and allowed to dry prior to the addition of cells. In illustrative embodiments, the transfection sheet is hydrated upon the addition of cells and cell culture media. In illustrative embodiments, the resilient sheet is positioned onto the cells. In illustrative embodiments, the pH and or temperature sensitivity of the lipid component of the nucleic acid-lipid composition results in a conformation change in the lipid component upon contact with the cultured cells that facilitates the transfer of nucleic acids to the target. For example, in illustrative embodiments, the melt temperature of the helper lipid is at or near the ambient temperature of the cultured cells. Warming of the lipid composition following the addition of cells to the culture dish causes a phase transition in the helper lipid that facilitates the release of nucleic acids from the transfection sheet and transfer of the nucleic acids to the target. In illustrative embodiments, the helper lipid phase transition is induced by the physiological pH of the transformation target. In illustrative embodiments, the helper lipid phase transition is induced by a combination of temperature and pH of the transformation target.

In some embodiments, the resilient sheets begins to break down and/or release nucleic acid at about physiological pH. In some embodiments, the resilient sheet breaks down in response to a change in the pH of cell culture media during a period of cell culture. In some embodiments, the resilient sheet begins to break down and/or release nucleic acid at about 37° C. In some embodiments, the resilient sheet maintains structural integrity in vivo, e.g., when applied to an organ or a tissue, for about 30 seconds, 1 minute, 2 minutes, 5 minutes, 10 minutes, 15 minutes, 30 minutes, 60 minutes, 2 hours, 3 hours, 6 hours, 12 hours or 24 hours or longer. Thus, in some embodiments, the resilient sheets provide a slow release of the nucleic acid to be transfected. In some embodiments, the resilient sheets maintain structural integrity in vitro (e.g., when applied to cells in culture) for about 30 seconds, 1 minute, 2 minutes, 5 minutes, 10 minutes, 15 minutes, 30 minutes, 60 minutes, 2 hours, 3 hours, 6 hours, 12 hours or 24 hours or longer. Thus, in some embodiments, cells to be transfected may undergo a variety of growth cycles or physiological transitions prior to or during transfection.

E. Kits

Also disclosed herein are kits for forming resilient sheets. In some embodiments, the kits include a cationic lipid and a helper lipid, and optionally instructions for combining the lipids to form resilient sheets with desired nucleic-acid release properties. In some embodiments, the kits include resilient sheets and optionally, instructions for transfecting a tissue, organ or cell with the resilient sheets.

### III. EXPERIMENTAL EXAMPLES

The present invention is further illustrated by the following examples, which should not be construed as limiting in any way.

### Example 1

### Preparation of a Resilient Transfection Sheet

The following components were used to form a resilient transfection sheet for nucleic acid transfer. The cationic lipid was 1,2-dioleoyl-3-trimethylammonium-propane (DOTAP; Avanti Polar Lipid, Inc.). The helper lipid was dioleoylphosphatidylethanolamine (DOPE; Avanti Polar Lipid, Inc.). The nucleic acid was CpG-DNA having the sequence TCCATGACGTTCCTGATGCT, SEQ ID NO: 1, (Bex Co., Ltd.). The nucleic acid stock solution was 1 mM in sterile water.

The sheet was prepared as follows:

At room temperature, 25 μl of the cationic lipid DOTAP was suspended in water to a final concentration of 20 mg/ml in an Eppendorf™ tube. To this, 7.84 μl of the 1 mM CpG-DNA solution was added. The ratio of DOTAP to nucleic acid was about 1 mol DOTAP to about 1-1.1 mol nucleic acid. The sample was mixed well upon addition of the CpG-DNA and then left stationary at room temperature for 30 to 60 minutes. During this period, a DOTAP/nucleic acid complex formed and was visible as a white precipitate. The solution was centrifuged at 10,000×g for 5 minutes and the supernatant was removed. The tube was inverted and the pellet allowed to dry.

The pellet was resuspended in 200 μl hexane (Wako Chemicals, USA; although other organic solvents e.g., methanol, ethanol, chloroform, hexane, etc. would also be appropriate), and 13.3 μl of 20 mg/ml DOPE was added. The ratio of cationic lipid to helper lipid was about 1 mol cationic lipid to about 0.1-1 mol helper lipid. The solution was then cast onto a Teflon™ sheet and dried overnight at room temperature. The dried sheet was then stored at room temperature until use.

For use in vitro, the lipid-nucleic acid solution was cast into a cell culture dish and dried overnight at room temperature. Cells were then cultured directly on the dried lipid-nucleic acid layer.

### Example 2

### Stimulation of IL-6 Production Using a CpG-DNA Transfection Sheet

BALB/c peritoneal macrophages were added to 96-well plates at a density of 2×10⁵ cells/well in 10% FBS, RPMI1640 (Sigma). Cells were grown under 5% CO₂ at 37° C. in a total volume of 200 and were stimulated with CpG-DNA either directly or with a CpG-DNA transfection sheet according to the following protocol:

Transfection sheet: A lipid-nucleic acid composition was prepared as described above, cast into the wells of a 96-well plate, and allowed to dry. Each well received a total of 0.2 nmol of CpG-DNA. Cells were added in a volume of 200 μl. The final concentration of CpG-DNA per well was 1 μM. After 18 hours of incubation, culture supernatants were collected and analyzed for IL-6 content using an OptEIA IL-6 ELISA kit (Becton Dickenson).

Direct stimulation: A 10 μM solution of CpG-DNA was prepared in 10% FBS, RPMI1640. Cells were added to the wells of a 96-well plate in a volume of 180 μl, together with 20 μl of CpG stock solution. The final concentration of CpG-DNA per well was 1 μM. After 18 hours of incubation, culture supernatants were collected and analyzed for IL-6 content using an OptEIA IL-6 ELISA kit (Becton Dickenson).

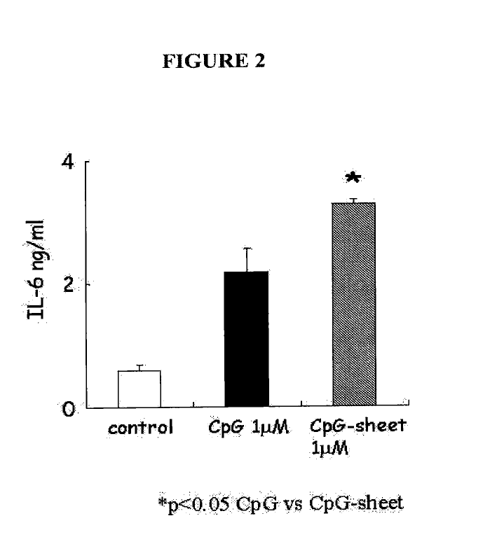

Results. As shown in FIG. 2, CpG-DNA sheets induced substantially higher IL-6 production than direct stimulation with CpG-DNA. Stimulation with a CpG gene transfer sheet induced 151% higher IL-6 production than direct stimulation with CpG. The CpG-DNA sheets also increased production of other cytokines in a similar fashion (data not shown).

### Example 3

### Transfection of Mammalian Tissues in Culture

This example will illustrate the use of gene transfer sheets for the transfection of mammalian tissues harvested and maintained in culture.

Transfection sheets: Four lipid-nucleic acid compositions will be prepared as described above, cast onto a sterile surface and allowed to dry. Three of the compositions will be prepared, each having an increasing amounts of a mammalian enzyme expression construct (e.g., 1×, 10× and 100×). The constructs will include a cDNA encoding an enzyme cloned into a mammalian expression vector. Such vectors are well known in the art. One of the compositions will be a control composition prepared using empty vector. Together, the four compositions will comprise a transfection set. Transfection sets will be prepared and tested in triplicate.

The gene transfer sheet will be shaped and sized to be smaller in area than the tissue sample targeted for transfection, with portions of the tissue not contacted by, and distant from, the gene transfer sheet serving as internal negative controls for enzyme expression.

Transfection of mammalian tissue: Mammalian tissues will be dissected from BALB/c mice and maintained in culture for 24-48 hours, using methods known in the art. To control for variability of transfection efficiency across tissue types and individual subjects, a single tissue or organ will be harvested and sectioned for use with each transfection set. Transfection sets will be applied to different tissue types to demonstrate the versatility of the gene transfection sheet. Gene transfer sheets will be applied to harvested tissues under standard culture conditions. At 24 hours post-transfection, the tissues will be prepared for whole-mount in situ hybridization using methods known in the art. Levels of enzyme mRNA will be detected and documented for each member of each transfection set. An exemplary experimental design is shown in the table below.

| Transfection Set | | |
|---|---|---|
| (relative amount of enzyme construct) | Tissue | Transfection time |
| A | Liver | 24 hours |
| (0, 1X, 10X, 100X) | | |
| B | Skeletal muscle | 24 hours |
| (0, 1X, 10X, 100X) | | |
| C | Epithelium | 24 hours |
| (0, 1X, 10X, 100X) | | |
| D | Neuronal | 24 hours |
| (0, 1X, 10X, 100X) | | |

Results: It is expected that transfection of mammalian tissues using gene transfer sheets will result in high-level expression of the transfected enzyme construct. No mRNA expression is expected in tissues transfected with the empty vector control. (“0”). For tissues transfected with the enzyme expression construct, it is expected that increasing amounts of the expression construct will correspond to increasing amounts of enzyme mRNA expression. Internal control tissues not contacted by the gene transfection sheet are expected to show no expression of the enzyme mRNA.

### Example 4

### In Situ Transfection of Mammalian Tissue

This example will illustrate the use of gene transfer sheets for the in situ transfection of mammalian tissue with an enzyme expression vector, such as in enzyme replacement therapy.

Transfection sheets: Four lipid-nucleic acid compositions will be prepared as described above, cast onto a sterile surface, and allowed to dry. Three of the compositions will be prepared each having an increasing amounts of a mammalian enzyme expression construct (e.g., 1×, 10× and 100×). The constructs will include a cDNA encoding an enzyme cloned into a mammalian expression vector. Such vectors are well known in the art. One of the compositions will be a control composition prepared using empty vector. Together, the four compositions will comprise a transfection set. Transfection sets will be prepared and demonstrated in triplicate. The gene transfer sheet will be smaller in area than the tissue targeted for transfection, with portions of the tissue not contacted by the gene transfer sheet serving as internal negative controls for enzyme expression, as described above.

Transfection of mammalian tissue: BALB/c mice will be anesthetized and a surgical incision made according to methods known in the art. Incisions will be made as necessary to provide access to tissues targeted for transfection. Each of the transfection sets will be applied to a different tissue type to demonstrate the versatility of the gene transfection sheet, with four animals. Gene transfer sheets will be applied to target tissues and the surgical incision closed. At 24 hours post-transfection, the target tissues will be harvested and prepared for whole-mount in situ hybridization using methods known in the art. Levels of enzyme mRNA will be detected and documented for targeted tissue. An exemplary experimental design is shown in the table below.

| Transfection Set | | |
|---|---|---|
| (relative amount of enzyme construct) | Tissue | Transfection time |
| A | Liver | 24 hours |
| (0, 1X, 10X, 100X) | | |
| B | Skeletal muscle | 24 hours |
| (0, 1X, 10X, 100X) | | |
| C | Epithelium | 24 hours |
| (0, 1X, 10X, 100X) | | |
| D | Neuronal | 24 hours |
| (0, 1X, 10X, 100X) | | |

Results: It is expected that transfection of mammalian tissues using gene transfer sheets will result in high-level expression of the transfected enzyme. No enzyme expression is expected in tissues transfected with the empty vector control. For tissues transfected with the enzyme expression construct, it is expected that increasing amounts of the expression construct will correspond to increasing amounts of enzyme expression. Internal control tissues not contacted by the gene transfection sheet are expected to show no expression of the enzyme.

The present disclosure is not to be limited in terms of the particular embodiments described in this application. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 proteins refers to groups having 1, 2, or 3 proteins. Similarly, a group having 1-5 proteins refers to groups having 1, 2, 3, 4, or 5 proteins, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

All references cited herein are incorporated by reference herein in their entireties and for all purposes to the same extent as if each individual publication, patent, or patent application was specifically and individually incorporated by reference in its entirety for all purposes.

1. A composition for cell transfection, the composition comprising:
a) one or more a cationic lipids;
b) one or more helper lipids; and
c) one or more nucleic acids;
wherein the composition is in the form of a resilient sheet.

2. The composition of claim 1, wherein the one or more helper lipids are neutral lipids.

3. The composition of claim 1, wherein the cationic lipid comprises an amphiphilic cationic lipid.

4. The composition of claim 1, wherein the cationic lipid is selected from the group consisting of: 1,2-dioleoyl-1,3-trimethylammonium-propane (DOTAP), 3β-[N-(N′,N′-dimethylaminoehtane)-carbomoyl]cholesterol (DC cholesterol), and combinations thereof.

5. The composition of claim 1, wherein the helper lipid selected from the group consisting of: dioleoylphosphatidylethanolamine (DOPE), dipalmitoylphosphatidylcholine (DPPC), distearoylphosphatidylcholine (DSPC), palmitoyloleoyl-phosphatidylcholine (POPC), palmitoyloleoyl-phosphatidylethanolamine (POPE), palmitoyloleyol-phosphatidylglycerol (POPG), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), egg phosphatidylcholine (EPC), or a combination thereof.

6. The composition of claim 1, wherein the helper lipid is dioleoylphosphatidylethanolamine (DOPE).

7. The composition of claim 1, wherein the nucleic acid comprises one or more of DNA, RNA, microRNA, siRNA, antisense DNA, plasmid DNA, aptamers, peptide nucleic acids, and CpG-DNA.

8. The composition of claim 1, wherein the molar ratio of cationic lipid to helper lipid is from about 1 to about 0.1-1.0.

9. The composition of claim 1, wherein the molar ratio of cationic lipid to nucleic acid is about 1 to about 1-1.1.

10. The composition of claim 1, wherein the resilient sheet comprises DOTAP and DOPE at a ratio of about 1 mole to about 0.1-1 mole.

11. A method for transfecting a cell, the method comprising: contacting the cell with the composition of claim 1.

12. The method of claim 11, wherein the cell is in vitro.

13. The method of claim 11, wherein the cell is in vivo.

14. The method of claim 11, wherein the cell is a mammalian cell.

15. The method of claim 11, wherein the cell is part of an organ.

16. A method of treating a subject with a therapeutic nucleic acid, the method comprising:
a) contacting the subject with the resilient sheet of claim 1, wherein the nucleic acid comprises a therapeutic nucleic acid.

17. The method of claim 16, wherein the therapeutic nucleic acid comprises a therapeutic siRNA, microRNA, an aptamer, or a combination thereof.

18. The method of claim 16, wherein the therapeutic nucleic acids encodes a therapeutic polypeptide.

19. The method of claim 16, wherein the resilient sheet contacts an organ of the subject.

20. The method of claim 16, wherein the resilient sheet contacts one or more of muscle tissue, epithelial tissue, connective tissue and nervous tissue of the subject.

21. The method of claim 16, wherein the resilient sheet contacts one or more of hematopoietic cells, epithelial cells and immune cell precursors of the subject.

22. A method of making a resilient sheet for cell transfection, the method comprising:
(a) combining a cationic lipid, a helper lipid and a nucleic acid to form a mixture;
(b) casting the mixture onto a support surface; and
(c) drying the cast solution thereby forming a resilient sheet.

23. The method of claim 22, further comprising: cutting the dried resilient sheet to a desired shape and or size.

24. The method of claim 22, wherein the ratio of cationic lipid to helper lipid is from about 1 to about 0.1-1.0.

25. The method of claim 22, wherein the ratio of cationic lipid to nucleic acid is about 1 to about 1-1.1.

26. The method of claim 22, wherein drying comprises evaporation at room temperature under ambient pressure.

27. The method of claim 22, wherein the cationic lipid is selected from the group consisting of: 1,2-dioleoyl-1,3-trimethylammonium-propane (DOTAP), 3β-[N-(N′,N′-dimethylaminoehtane)-carbomoyl]cholesterol (DC cholesterol), and combinations thereof.

28. The method of claim 22, wherein the helper lipid is selected from the group consisting of: dipalmitoylphosphatidylcholine (DPPC), distearoylphosphatidylcholine (DSPC), palmitoyloleoyl-phosphatidylcholine (POPC), palmitoyloleoyl-phosphatidylethanolamine (POPE), palmitoyloleyol-phosphatidylglycerol (POPG), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), egg phosphatidylcholine (EPC) or a combination thereof.

29. The method of claim 22, wherein the nucleic acid comprises one or more of: DNA, RNA, microRNA, siRNA, antisense DNA, plasmid DNA, aptamers and CpG-DNA.

30. A kit comprising:
(a) the composition of claim 1; and
(b) instructions for use.


SEQUENCE LISTING (1 sequence)

SEQ ID NO: 1 - DNA, 20 bp, Artificial Sequence
  Description of Artificial Sequence Synthetic oligonucleotide
tccatgacgttcctgatgct